(19) United States
(12) Patent Application Publication
(10) Pub. No.: US 2021/0361838 A1
(43) Pub. Date: Nov. 25, 2021

(54) BREAST PUMP AND FEEDING KIT

(71) Applicant: THINK GREEN LIMITED, Auckland (NZ)

(72) Inventor: Shu Ting Zhang, Auckland (NZ)

(21) Appl. No.: 16/332,722

(22) PCT Filed: Nov. 30, 2018
(86) PCT No.: PCT/NZ2018/050171

§ 371 Date: Mar. 12, 2019

(30) Foreign Application Priority Data
Nov. 30, 2017 (NZ) 737838

(51) Int. Cl. A61M 1/06; A61J 9/00; A61J 11/00; A61J 11/02; A61J 11/04; A61J 9/06

(52) U.S. Cl. CPC A61M 1/068; A61M 1/064; A61J 9/005; A61J 9/0623; A61J 11/008; A61J 11/02; A61J 11/045; A61J 9/006

(57) ABSTRACT

A breast pump (1) comprises a cup member (2) and a container (3). The cup member (2) has a breast engaging portion (4) configured to seal to a breast. The container (3) is configured to be removably and sealingly connected to the cup member (2). The container (3) is configured to be deformed by a user and released to create suction on the breast to pump breast milk into the container (3). A breast pump (1) and feeding kit for pumping, storing and feeding breast milk is also disclosed. The kit comprises a deformable container (3) which may be attached to one of a cup member (2), a teat (18), or a lid (32). The teat (18) is configured to feed breast milk from the container (3) to a baby. The lid (32) is configured to secure the breast milk in the container for storage.

### FIELD OF INVENTION

The present invention relates to a device for obtaining breast milk. Particularly, but not exclusively, the present invention relates to a breast pump and feeding kit.

### BACKGROUND OF THE INVENTION

Many mothers prefer to breast feed instead of using powdered milk formulas for feeding babies. The American Academy of Pediatrics recommends exclusive breastfeeding for the first six months of a baby's life and breastfeeding with the introduction of foods for at least the first year of a baby's life to ensure the short and long-term health of the baby. However, there are situations where breastfeeding may not be possible or practical, for instance if the mother is unwell, working, travelling, or running errands. Breastfeeding may also be difficult if the baby is not latching well or if the mother has undergone breast surgery and is unable to be stimulated by the suckling reflex. In these cases, it is still possible to provide the baby with breast milk through the use of breast pumps.

Breast pumps are devices which allow mothers to express breast milk that can then be stored and used at a later time. There are various different types of breast pumps including manual and electric pumps.

An example of a manual breast pump may include a hand pump, a breast shield for sealing to the mother's breast, and a bottle. The hand pump may comprise a handle and a diaphragm assembly. Moving the handle back and forth will move the diaphragm and create suction that will induce the expression of milk. Existing manual breast pumps may be bulky and may have valves and other complex mechanisms that may be difficult to clean and/or assemble. They may also be tiring to use.

An electric pump may operate in a similar way, however in place of a handle that must be manually moved up and down, the pump mechanism is driven by an electric motor. Electric pumps have the disadvantage of requiring a power source, which may limit where can be used. An electric pump may have a rechargeable battery which would allow for it to be transported and used in more locations, however this would require that the battery is kept charged and may not last the whole day. The motor and battery may also be heavy or bulky which could make transport difficult. Electric pumps may also be expensive.

It is an object of the present invention to provide a device or kit for obtaining and/or storing breast milk that addresses or ameliorates at least one of the aforementioned needs/problems.

It is an alternative object of the present invention to provide a device or kit for obtaining breast milk and feeding the breast milk to an infant that addresses or ameliorates at least one of the aforementioned needs/problems.

Alternatively, it is an object of the present invention to at least provide the public with a useful choice.

### SUMMARY OF THE INVENTION

According to a first aspect of the invention, there is a provided a breast pump, comprising:
a cup member having a breast engaging portion configured to seal to a breast; and
a container configured to be removably and sealingly connected to the cup member,
wherein the container is configured to be deformed by a user and released to create suction on the breast to pump breast milk from the breast into the container.

According to a second aspect of the invention, there is provided a breast pump and feeding kit for pumping, storing, and feeding of breast milk, the kit comprising:
a cup member having a breast engaging portion configured to seal to a breast;
a container; and
a teat,
   wherein the cup member and the container are configured to be removably and sealingly connected together and the container is configured to be deformed by a user and released to create suction on the breast to pump breast milk from the breast into the container; and
   wherein the teat and the container are configured to be removably and sealingly connected together for feeding breast milk from the container to a baby.

Preferably the container is made from an elastic material which returns to its original shape and size after deformation.

Preferably, the breast pump and feeding kit comprises a ring member configured to connect the cup member to the container. Preferably, the ring member is substantially rigid.

Preferably, the teat is configured to connect to the ring member.

Preferably, the container comprises a rim comprising a screw thread to connect to a complementary screw thread on the ring member.

Preferably, the ring member comprises a tab extending from a side of the ring member, the tab configured to assist the user in screwing the ring member on to or off the container.

Preferably, the ring member comprises a connection flange and the cup member comprises a circumferential groove configured to receive the connection flange. Alternatively, the cup member comprises a connection flange and the ring member comprises a circumferential groove configured to receive the connection flange.

Preferably, the cup member comprises an end groove configured to fit to the container and seal therewith. Preferably, the end groove is configured to fit to the rim of the container.

Preferably, the teat comprises a circumferential groove configured to receive the connection flange of the ring member. Alternatively, the teat comprises a connection flange and the ring member comprises a circumferential groove configured to receive the connection flange.

Preferably, the teat comprises an end groove configured to fit to the container and seal therewith. Preferably, the end groove is configured to fit to the rim of the container.

Preferably, the breast pump and feeding kit comprises a teat cover configured to connect to and at least partially cover the teat.

Preferably, the container comprises one or more indentations in an outer surface of the container configured to facilitate gripping of the container.

Preferably, the container is formed from silicone.

Preferably, the container is integrally formed.

Preferably, the teat is integrally formed.

Preferably, the breast engaging portion of the cup member is substantially frustoconical in shape.

Preferably, breast engaging portion comprises a channel configured to direct a flow of milk when poured from the container.

Preferably, the cup member comprises a neck portion connected to the breast engaging portion, the neck portion being narrower than the breast engaging portion.

Preferably, the neck portion is substantially cylindrical in shape.

Preferably, the breast engaging portion opens at an oblique angle to an axis of the container.

Preferably, the cup member comprises a connecting portion configured to connect to the container.

Preferably, the connecting portion is provided at an end of the neck portion opposite the breast engaging portion.

Preferably, the cup member is formed from a flexible material. More preferably the cup member is formed from silicone.

Preferably, the cup member is integrally formed.

According to a third aspect of the invention, there is provided a breast pump comprising:
a cup member having a breast engaging portion configured to seal to a breast; and
a container configured to be removably and sealingly connected to the cup member,
wherein the container is configured to be deformed by a user and released to create suction on the breast to pump breast milk from the breast into the container, and
wherein the container is configured to be removably and sealingly connected to a teat for feeding breast milk from the container to a baby.

Preferably the container is made from an elastic material which returns to its original shape and size after deformation.

Preferably, the breast pump comprises a ring member configured to connect the cup member to the container. Preferably, the ring member is substantially rigid.

Preferably, the container comprises a rim comprising a screw thread to connect to a complementary screw thread provided on the ring member.

Preferably, the ring member comprises a tab extending from a side of the ring member, the tab being configured to assist the user in screwing the ring member on to or off of the container.

Preferably, the ring member comprises a connection flange and the cup member comprises a circumferential groove configured to receive the connection flange. Alternatively, the cup member comprises a connection flange and the ring member comprises a circumferential groove configured to receive the connection flange.

Preferably, the cup member comprises an end groove configured to fit to the container and seal therewith. Preferably, the end groove is configured to fit to the rim of the container.

Preferably, the ring member is configured to connect to the teat. Preferably, the connection flange of the ring member is configured to connect to the teat.

Preferably, the container is configured to seal to the teat. Preferably, the rim of the container is configured to seal to a groove of the teat.

Preferably, the container comprises one or more indentations in an outer surface of the container, the one or more indentations being configured to enable the bottle to be gripped.

Preferably, the container is formed from silicone.

Preferably, the container is integrally formed.

Preferably, the breast engaging portion of the cup member is substantially frustoconical in shape.

Preferably, breast engaging portion comprises a channel configured to direct a flow of milk when poured from the container.

Preferably, the cup member comprises a neck portion connected to the breast engaging portion, the neck portion being narrower than the breast engaging portion.

Preferably, the neck portion is substantially cylindrical in shape.

Preferably, the breast engaging portion opens at an oblique angle to an axis of the container. Preferably, the cup member comprises a connecting portion configured to connect to the container, the connecting portion provided at an end of the neck portion opposite the breast engaging portion.

Preferably, the cup member is formed from a flexible material. More preferably the cup member is formed from silicone.

Preferably, the cup member is integrally formed.

According to a fourth aspect of the invention, there is provided a breast pump comprising:
a cup member having a breast engaging portion configured to seal to a breast;
a container configured to be removably and sealingly connected to the cup member, and
a ring member configured to connect the cup member to the container,
wherein the container is configured to be deformed by a user and released to create suction on the breast to pump breast milk from the breast into the container.

Preferably, the ring member is substantially rigid.

Preferably, the container comprises a rim comprising a screw thread to connect to a complementary screw thread on the ring member.

Preferably, the ring member comprises a tab extending from a side of the ring member, the tab configured to assist the user in screwing the ring member on to or off the container.

Preferably, the ring member comprises a connection flange and the cup member comprises a circumferential groove configured to receive the connection flange. Alternatively, the cup member comprises a connection flange and the ring member comprises a circumferential groove configured to receive the connection flange.

Preferably, the cup member comprises an end groove configured to fit to the container and seal therewith. Preferably, the end groove is configured to fit to the rim of the container.

Preferably, the ring member is configured to a teat to removably and sealingly connect the container and the teat for feeding breast milk from the container to a baby.

According to a fifth aspect of the invention, there is provided a breast pump comprising:
a cup member having a breast engaging portion configured to seal to a breast about an opening of the breast engaging portion; and
a container configured to be removably and sealingly connected to the cup member,
wherein the container is configured to be deformed by a user and released to create suction on the breast to pump breast milk from the breast into the container, and
wherein the breast engaging portion opens at an oblique angle to an axis of the container.

Preferably, the breast pump comprises a ring member configured to connect the cup member to the container. Preferably the ring member is substantially rigid.

Preferably, the container is substantially cylindrical about the axis of the container.

Preferably, the container comprises a rim comprising a screw thread to connect to a complementary screw thread on the ring member.

Preferably, the ring member comprises a tab extending from a side of the ring member, the tab configured to assist the user in screwing the ring member on to or off the container.

Preferably, the ring member comprises a connection flange and the cup member comprises a circumferential groove configured to receive the connection flange. Alternatively, the cup member comprises a connection flange and the ring member comprises a circumferential groove configured to receive the connection flange.

Preferably, the cup member comprises an end groove configured to fit to the container and seal therewith. Preferably, the end groove is configured to fit to the rim of the container.

Preferably, the ring member is configured to a teat to removably and sealingly connect the container and the teat for feeding breast milk from the container to a baby.

Preferably, the breast engaging portion of the cup member is substantially frustoconical in shape.

Preferably, breast engaging portion comprises a channel configured to direct a flow of milk when poured from the container.

Preferably, the cup member comprises a neck portion connected to the breast engaging portion, the neck portion being narrower than the breast engaging portion.

Preferably, the neck portion is substantially cylindrical in shape about an axis aligned with the axis of the container.

Preferably, the cup member comprises a connecting portion configured to connect to the container, the connecting portion provided at an end of the neck portion opposite the breast engaging portion.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 is a cross-sectional side view illustration of a breast pump according to an embodiment of the invention;

FIG. 2 is an exploded cross-sectional view illustration of the breast pump of FIG. 1;

FIG. 3 is a perspective side view illustration of the cup member of the breast pump of FIG. 1;

FIG. 4 is a perspective view illustration from the side and front of the cup member of the breast pump of FIG. 1;

FIG. 5 is a side view illustration of the breast pump of FIG. 1;

FIG. 6 is an exploded side view illustration of a container, ring, teat and cover of a breast pump and feeding kit forming a bottle for feeding collected milk to a baby according to an embodiment of the invention;

FIG. 7 is a cross-sectional view illustration of the container, ring and teat of FIG. 6;

FIG. 8 is an exploded side view illustration of a container, ring, sipping straw, handles, and cover of a breast pump and feeding kit forming a bottle for feeding collected milk to a baby according to an embodiment of the invention;

FIG. 9 is a perspective view illustration of the sipping straw of FIG. 8;

FIG. 10 is an exploded side view illustration of a container, ring, spoon attachment, and cover of a breast pump and feeding kit forming a bottle for feeding food to a baby according to an embodiment of the invention;

FIG. 11 is a perspective view illustration of the spoon attachment of FIG. 10;

FIG. 12 is a side view illustration of a container, ring, and lid of a breast pump and feeding kit forming a storage container for storing collected milk according to an embodiment of the invention;

FIG. 13 is a perspective view illustration of the lid of FIG. 12.

### BRIEF DESCRIPTION OF PREFERRED EMBODIMENTS OF THE INVENTION

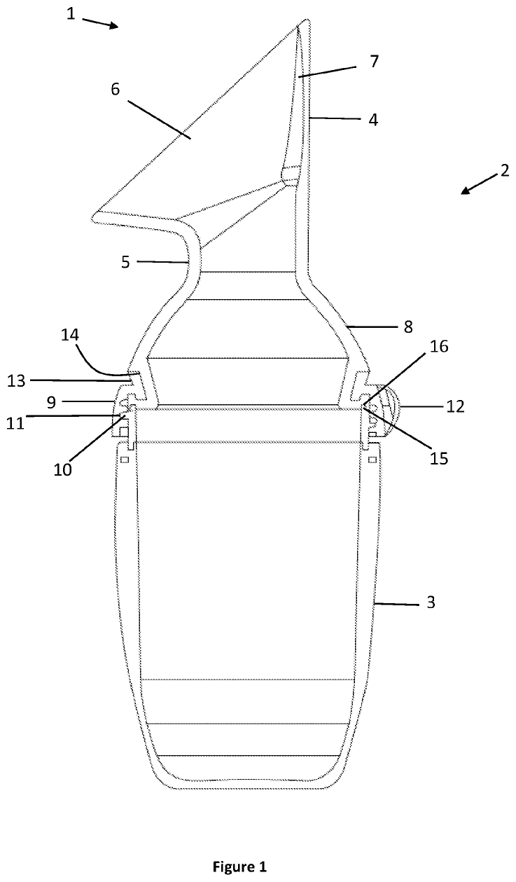

Referring first to FIGS. 1, 2 and 5, a breast pump for use in pumping breast milk according to a preferred embodiment of the invention, is generally referenced by arrow 1. The breast pump 1 comprises a cup member, generally referenced by arrow 2, and a container 3.

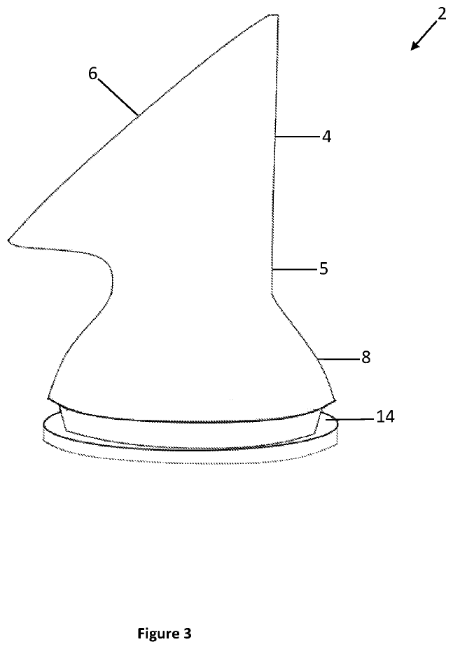

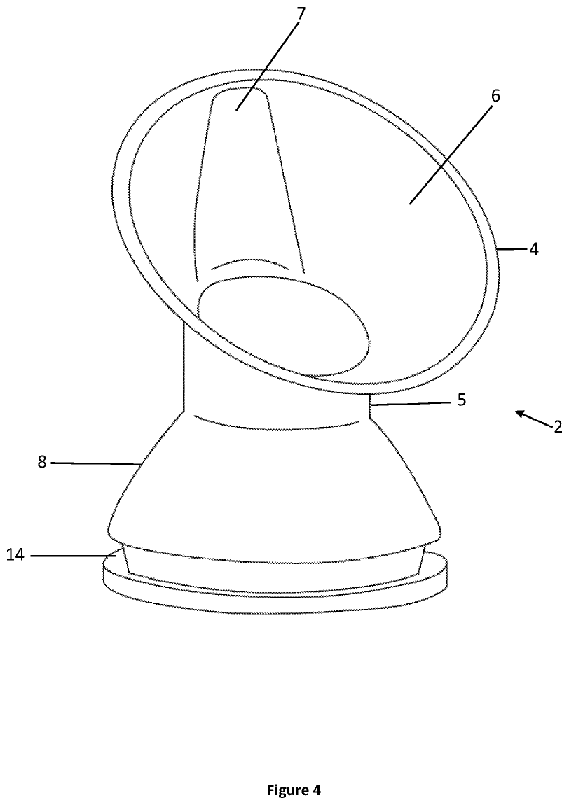

Referring next to FIGS. 3 and 4 in particular, the cup member 2 comprises a breast engaging portion 4 configured in use to form an air tight seal with a mother's breast. In this embodiment, the breast engaging portion includes an opening 6 to receive a portion of a breast and to receive expressed breast milk. The breast engaging portion 4 may be of any suitable size and shape to form a sufficient and stable seal with a breast to enable suction to be applied to the breast. In this embodiment, the breast engaging portion 4 is frustoconical in shape. In alternative embodiments, the breast engaging portion 4 may be hemispherical in shape, breast-shaped, horn-shaped, or have a planar flange at the end. The breast engaging portion 4 may alternatively have a shape similar to the shape of a baby's mouth.

In the embodiment shown in the figures, the breast engaging portion 4 is formed by a frustoconically-shaped wall. The breast engaging portion 2 may also be identified as a cushion or a breast shield. The breast engaging portion 4 may be made of a soft, flexible material. In some forms of the technology the entire cup member 2 is integrally formed of a flexible material. A cup member which is integrally formed of a flexible material may provide ample flexibility to conform to a range of breast shapes and sizes and achieve a stable and comfortable seal for a range of users. In particular, flexibility in the cup member 2, including at the lower end, may assist in forming a stable seal to a wide range of users' breasts. In a preferred embodiment the cup member 2 is formed from silicone, although in alternative embodiments the cup member 2 may be formed from another biocompatible material, such as a thermoplastic elastomer, rubber, gel, closed cell foam, or flexible plastic.

The container 3 may also be formed from silicone or from another soft, flexible material, such as a rubber, thermoplastic elastomer or flexible plastic. In preferred embodiments, the container 3 is formed from an elastically deformable material which returns to its original shape and size after deformation. In other embodiments, the container may comprise a non-elastic material biased towards its original shape and size by a spring element. The container 3 is configured to be deformed by the user (e.g. squeezed) and then released. When the container 3 is attached to the cup member 2 and is in use as a breast pump this deformation results in a change of volume inside the container. The deformation reduces the volume of the container 3 and forces air out of the container, breaking the seal at the breast engaging portion 4. When the container 3 is released, the container 3 returns to its original shape, creating suction on the breast at the breast engaging portion 4 when the breast engaging portion 4 is engaged to a breast. The suction results in an expression of breast milk which is collected inside the container 3.

The breast engaging portion 4 may be configured such that the opening 6 is provided at an oblique angle to an axis of the container 3. In a preferred embodiment, the container 3 may be generally cylindrical in shape, and the opening 6 is provided at an oblique angle to a central axis and/or a longitudinal axis of the container 3. The opening 6 of the breast engaging portion may also be provided at an oblique angle to an opening of the container. The angled opening 6 with respect to the container 3 enables the container 3 to be located lower than the user's breast, and oriented close to an upright position, during use. This may be a comfortable orientation for the user to use the breast pump 1 and may reduce the risk of spilling breast milk during or after collection.

The breast engaging portion 4 of the cup member 2 may comprise a channel 7, best seen in FIG. 4, for directing the milk if the milk is poured out of the container 3 through the opening 6 in the breast engaging portion 4. The channel 7, in this embodiment, is an elongate recessed portion formed into the wall of the breast engaging portion 4.

The cup member 2 further comprises a neck portion 5 and a connecting portion 8. The neck portion 5 is connected to the breast engaging portion 4 on an opposite side to the opening 6. The frustoconically shaped wall forming the breast engaging portion 4 extends from an end of the neck portion 5. The neck portion is hollow to receive milk from the breast engaging portion 4. In the embodiment shown, the neck portion 5 is generally cylindrical in shape. In other embodiments, the neck portion 5 may have a different shape, such as a frustoconical shape. In the embodiment shown, the neck portion 5 is narrower than the breast engaging portion 4. In the preferred embodiment, the neck portion 5 is cylindrical about an axis aligned with a longitudinal axis of the container 3.

The connecting portion 8 of the cup member 2 is connected to the neck portion 5 at the opposite end of the cup member 2 to the breast engaging portion 4. The connecting portion 8 is configured to removably attach to the container 3. In this embodiment, the connecting portion 8 is formed by a curved wall extending outwardly from the neck portion 5 around the circumference of the neck portion 5. One end of the connecting portion 8 is connected to the neck portion 5 and the other end is configured to connect to the container 3. The neck portion 5 and connecting portion 8 may be made of a soft, flexible material, and in this embodiment are formed from silicone. In alternative embodiments, the neck portion 5 and connecting portion 8 may be formed from a material such as rubber, thermoplastic elastomer, closed-cell foam, gel, or a flexible plastic. The breast engaging portion 4, the neck potion 5, and connecting portion 8 of the cup member 2 are preferably integrally formed. In other embodiments of the invention, the parts may be formed separately and may be permanently or removably connected to one another.

In other embodiments of the invention (not shown), the breast pump may not include a neck portion or a separate connecting portion. In such embodiments, the opposite side of the breast engaging portion to the opening may be configured to connect directly to the container.

In the embodiment shown in the figures, the breast pump 1 comprises a ring member 9 which provides an attachment means by which the cup member 2 and container 3 are able to be removably connected. The ring member 9 is configured to connect the cup member 2 to the container 3. In preferred embodiments the ring member 9 is comprised of a substantially rigid material such as polypropylene, polycarbonate, polyethylene, nylon, a metal suitable for contact with food such as aluminium or stainless steel, or glass. The ring member 9 may be less rigid in alternative embodiments of the invention, but in preferred embodiments the ring member 9 adds rigidity to the container 3 and cup member 2 and creates a robust connection.

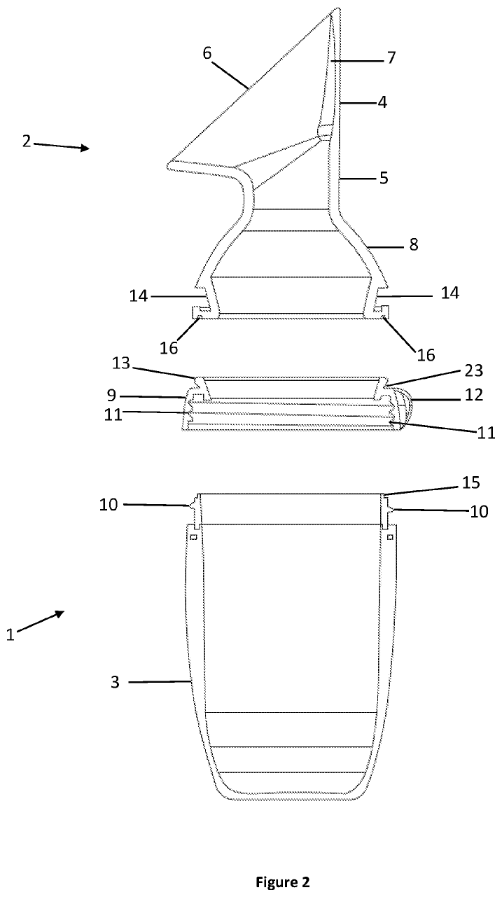

FIG. 2 shows an exploded view of the breast pump 1, including the ring member 9. In this example of the technology the ring member 9 attaches to the container 3 by way of a screw thread. The container 3 may comprise a first threaded portion 10 at a top rim to connect to a complementary second threaded portion 11 of the ring member 9. The first threaded portion 10 and second threaded portion 11 may comprise complementary screw threads cut or formed in the material from which the ring member 9 and container 3 are formed. In the embodiment shown the container 3 comprises an external screw thread 10 provided on an outer surface of the container 3. The ring member 9 comprises an internal screw thread 11 provided on an inner surface of the ring member 9. The first threaded portion 10 is configured to be screwed to the second threaded portion 11 to make a sealed connection which is secure and leak-proof. In alternative embodiments (not shown), the first threaded portion 10 on the container 3 may be an internal screw thread, and the second threaded portion 11 on the ring member 9 may be an external screw thread. In alternative embodiments, the breast pump may comprise a ring member which connects to the container 3 in another manner, such as by way of a friction fit, flange-groove connection, releasable snap-fit, bayonet fitting and the like.

The ring member 9 may comprise a tab 12. In the embodiment shown, the tab 12 extends from an outer side surface of the ring member 9. The tab 12 may be configured to assist the user to screw the ring member 9 on to or off the container 3. The tab 12 provides a contact point for the user to place their finger and/or thumb to provide force when removing or connecting the ring member 9 to the container 3. In other embodiments, the tab 12 may be replaced by a pair of handles configured to help the user to screw on the ring member and also to enable the baby to hold the container 3 while feeding.

The ring member 9 further comprises a connection flange 13 which is configured to connect to a circumferential groove 14 in the cup member 2. In the embodiment shown, the circumferential groove 14 is provided at the end of the connecting portion 8 of the cup member, opposite the breast engaging portion 4.

To connect the cup member 2 to the ring member 9, the connecting portion 8 of the cup member 2 may be deformed and the circumferential groove 14 is fitted over and engages the connection flange 13, as shown in FIG. 1, to form a substantially secure and leak-proof seal. The cup member 2 may be removed by deforming the connecting portion 8 and removing the connection flange 13 of the ring member 9 from the circumferential groove 14. The connection flange 13 may be provided at an angle relative to the sides of the container 3. The connection flange 13 may be angled outwardly with respect to the container 3. The angle of the connection flange 13 may aid in securing the cup member 2 to the ring member 9 and may assist in preventing unintended disconnection during use.

The complementary groove 14 of the connecting portion 8 is angled with respect to a central axis of the connecting portion 8 to complement the angle of the connection flange 13. In alternative forms of the technology (not shown) the connection flange 13 may be provided on the connecting portion 8 and the circumferential groove 13 may be formed in the ring member 9. In alternative embodiments the breast pump may comprise a ring member which connects to the cup member 2 in another manner, such as by way of a friction fit, releasable snap-fit, threaded connection and the like.

The container 3 comprises a rim 15, as best seen in FIGS. 1 and 2. The rim 15 may be configured to engage a corresponding end groove 16 of the connection portion 8 of the cup member 2. Accordingly, the end groove 16 may fit to the rim 15 of the container 3 and form a seal therewith. This connection creates a seal directly between the cup member 2 and the container 3, which may aid in ensuring that the breast milk does not leak as it flows between the cup member 2 and the container 3. This type of connection may also improve the airtightness of the connection, which may be important for creating suction at the user's breast. When the cup member 2 and container 3 are formed exclusively from silicone, this connection also prevents the milk from contacting any other material than silicone, such as the material from which the ring member 9 is formed. Even when the ring member 9 is formed from a material suitable for contact with food, some users may prefer that the milk only contacts silicone. Additionally, the compliance and resilient properties of silicone may provide for a robust seal between the cup member 2 and container 3.

In alternate forms of the technology (not shown), the breast pump may not include a ring member and the container 3 may connect directly to the cup member 2 by another attachment means. For example, the rim 15 of the container 3 may include a connection flange for connecting to the connecting portion 8 of the cup member 2, enabling the container 3 to connect directly to the circumferential groove 14 of the connecting portion 8. In some forms of the technology the connecting portion 8 may not be present and the container 3 or the ring member 9 may connect directly to the neck portion 5, with an attachment means in the form of a threaded or friction fit connection.

In some forms of the technology, the top rim of the container 3 that connects to the ring member 9 and cup member 2 may be formed of a substantially rigid material. The rigid material may be overmoulded to the material forming the remainder of the container 3. A rigid material for the rim may form a better seal with the resilient material from which the cup member 2 is formed.

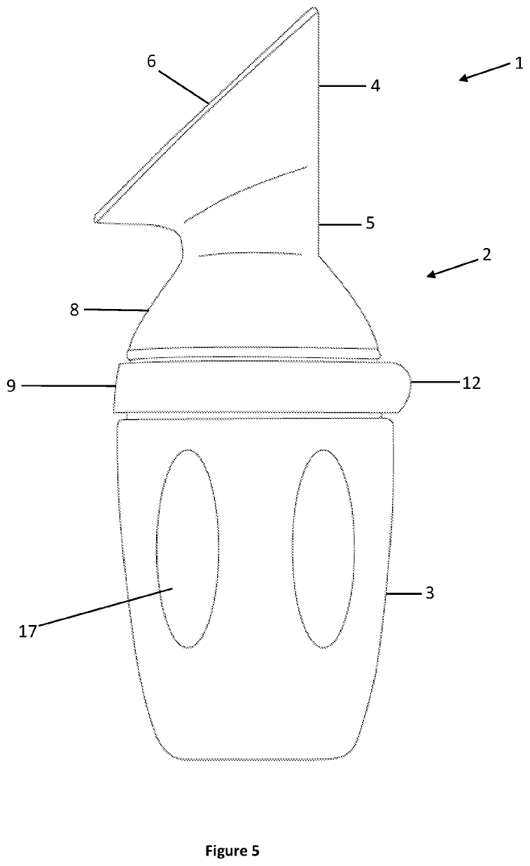

As best seen in FIG. 5, in the embodiment shown the container 3 comprises one or more indentations 17 on the outside surface of the container 3 in order to make the container 3 easier to hold and/or deform by the user. In some forms of the technology, the container 3 is transparent, and may comprise one or more graduations for indicating the amount of milk inside.

In some forms of the technology, the container 3 may comprise a suction cup (not shown) either formed on or attached to the bottom of the container 3. A suction cup allows for the container 3 to be removably attached to a surface such as a table or bench via suction, which helps prevent the milk from spilling after it is collected in the container 3.

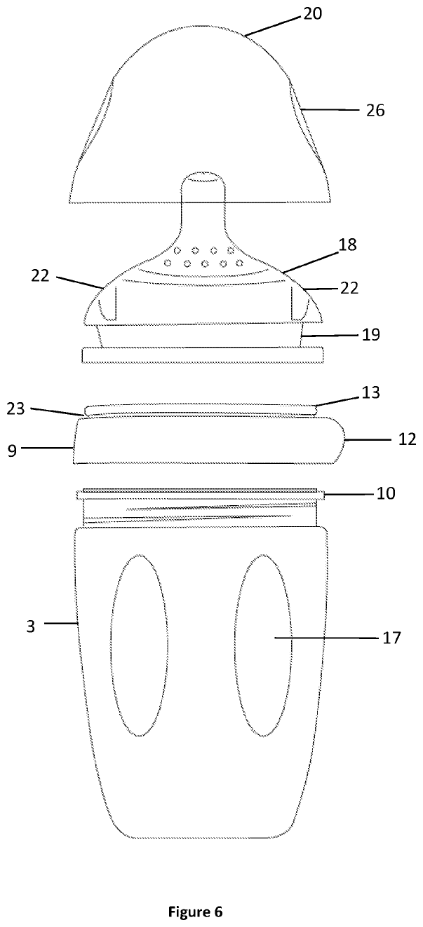

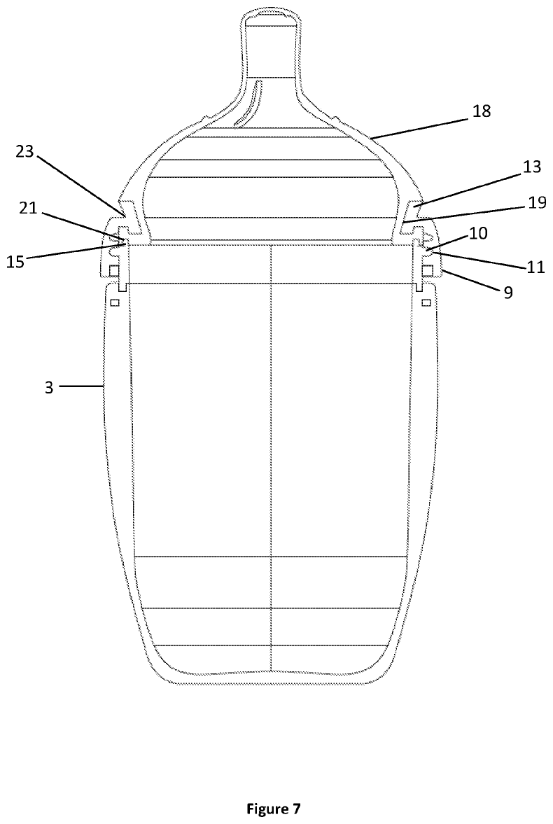

The cup member 2 and container 3 of the breast pump 1 may form part of a breast pump and feeding kit comprising at least one teat 18, as shown in FIGS. 6 and 7. The teat 18 is configured for feeding breast milk to a baby. Once the breast milk has been obtained using the breast pump 1, the cup member 2 may be separated from the container 3 and the teat 18 may be connected to the container 3. The container 3 and teat 18, once connected, form a bottle which is able to be used for feeding or storage of breast milk.

In a preferred form of the technology, the teat 18 comprises a complementary groove 19 which is configured to fit over the connection flange 13 of the ring member 9 to form a sealed connection which is substantially leak-proof. The teat 18 may be made of a flexible material, such as silicone. A lower portion of the teat 18 may be deformable to enable the circumferential groove 19 to engage the connection flange 13 to connect the teat 18 and container 3. The teat 18 can be separated from the container by deforming the teat 18 and disengaging the connection flange 13 from the circumferential groove 19. In the preferred embodiment, the teat 18 connects to the container 3 in the same manner as the cup member 2 attaches to the container 3.

In a preferred form of the technology the rim 15 of container 3 may be configured to engage a corresponding end groove 21 of the teat 18. Accordingly, the end groove 21 may fit to the rim 15 of the container 3 and form a seal therewith. This connection forms a seal directly between the teat 18 and the container 3, which may aid in ensuring that the breast milk does not leak as it flows between the container 3 and the teat 18. When the teat 18 and container 3 are integrally formed from silicone, this connection also prevents the milk from contacting any other material than silicone, such as the material from which the ring member 9 is formed. Even when the ring member 9 is formed from a material suitable for contact with food, some users may prefer that the milk only contacts silicone. Additionally, the compliance and resilient properties of silicone may provide for a robust seal between the teat 18 and container 3.

The teat 18 may also include one or more vent holes 22. The holes 22 are configured to allow air to vent between the surroundings and the container 3. This aids in ensuring that the teat 18 does not suck inwards during feeding. It also helps to prevent the child swallowing air which can cause colic.

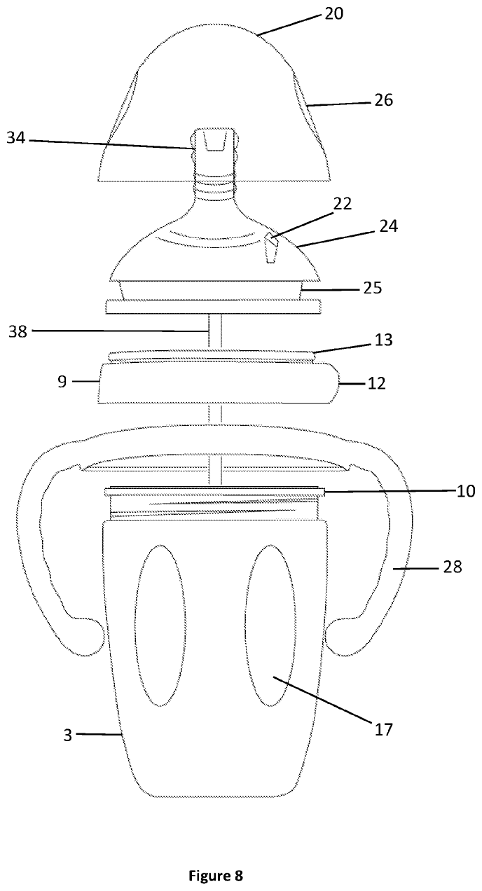

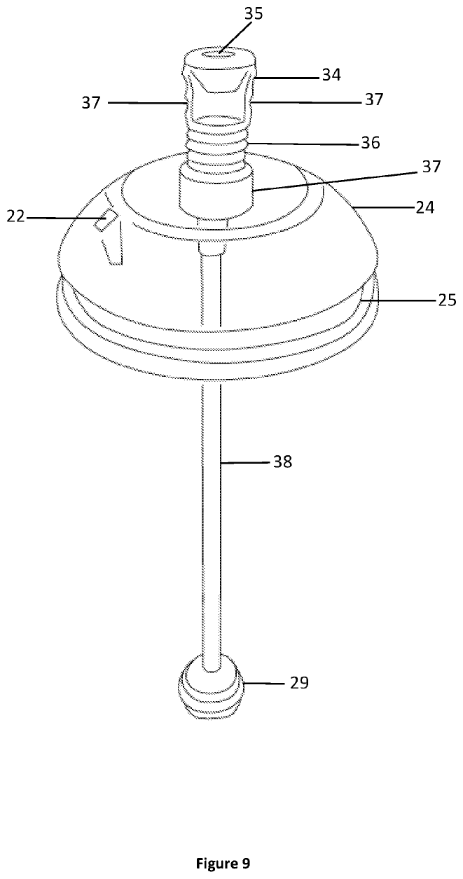

In some forms of the technology, the breast pump and feeding kit may also include a sipping straw attachment 24, as shown in FIGS. 8 and 9. In the embodiment shown the sipping straw attachment 24 is transparent. The sipping straw attachment 24 is configured for feeding breast milk or other liquids to a baby. Similar to the use of the teat, once the breast milk has been obtained using the breast pump 1, the container 3 can be separated from the cup member 2 and connected to the sipping straw attachment 24. Once connected the sipping straw attachment 24 and the container 3 form a bottle which can be used for feeding. The sipping straw attachment 24 may be suited for feeding slightly older children compared to the teat. In a preferred form of the technology, the sipping straw attachment 24 comprises a complementary groove 25 which engages the connection flange 13 of the ring member 9 to form a sealed connection which is substantially leak-proof. As with the teat 18, the sipping straw attachment 24 may also be made of a flexible material, such as silicone, and may also be deformable to enable attachment with the connection flange 13. The container 3 may also be provided with a handle attachment 28 which may aid the child in using the bottle. The handle attachment 28 may have a circular opening such that it can be fitted around the top rim of the container 3 and below the ring member 9 when connected. The sipping straw attachment 24 may comprise a nozzle 34, with an aperture 35, which the infant can use to draw liquid from the container 3. The nozzle 34 may have grooves 37 to make it easier for the infant to grip the nozzle 34. The nozzle 34 may also have a flexible section 36 to make it easier for the infant to grip the nozzle 34 even when the bottle is held at different angles. The sipping straw attachment 24 further comprises a connecting section which connects the nozzle 35 to the straw 38. The straw is preferably made from a flexible material. As shown in FIG. 9, a weight 29 may be provided at the end of the straw 38 to ensure that the end of the straw 38 is submerged in the liquid held by the container 3 at all times, even when the container 3 is held at different angles or upside down. The sipping straw attachment 24 may also include one or more vent holes 22 similar to those for the teat 18 described above.

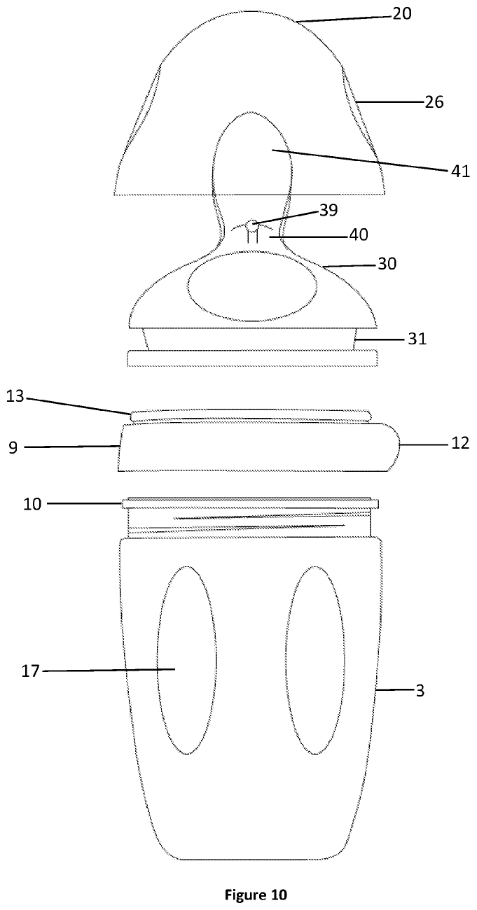

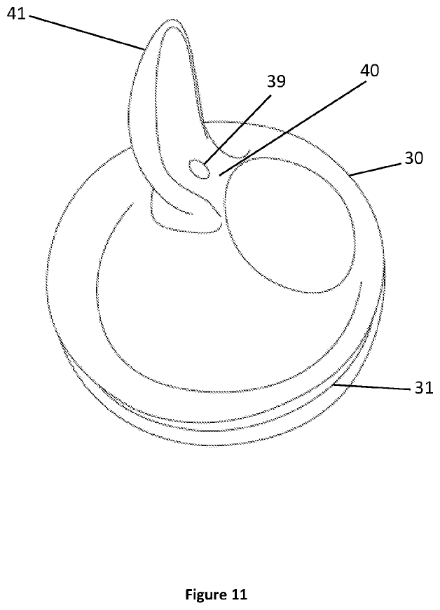

In some forms of the technology, the breast pump and feeding kit may also include a spoon attachment 30, as shown in FIGS. 10 and 11. The spoon attachment 30 is configured for feeding the baby and can be used with liquid foods, preferably viscous foods such as yoghurt and purees. In a preferred form of the technology, the spoon attachment 30 comprises a complementary groove 31 which is configured to engage the connection flange 13 of the ring member 9 to form a sealed connection which is substantially leak-proof. As with the teat 18 and the sipping straw 24, the spoon attachment 30 may also be made of a flexible material, such as silicone, and may also be deformable to enable attachment with the connection flange 13.

The spoon attachment may be provided with an aperture 39 which extends from an interior surface of the connecting portion of the spoon attachment to the spoon formation 41, more preferably to the base 40 of the spoon formation 41. Liquid food held in the container can be made to flow through the aperture 39 to the spoon formation 41 by squeezing the sides of the container.

The alternative means for attachment described above in relation to the cup member 2 and container 3 may also be utilised in connecting the teat 18, sipping straw 24, or spoon 30 and the container 3. For example, the container 3 may connect directly to the teat 18, sipping straw 24, or spoon 30. Or the teat 18, sipping straw 24, or spoon 30 may screw on to a ring member, in alternative embodiments. The teat 18, sipping straw 24, or spoon 30 may comprise a substantially rigid portion (e.g. a rigid overmoulded portion) configured for connection to the ring member or to the container 3.

The breast pump and feeding kit may also include a cover 20 (show in FIGS., 6, 8, and 10) configured to cover the teat 18, sipping straw 24, or spoon 30 when the bottle is not in use. The cover 20 may connect to teat 18, sipping straw 24, or spoon 30 to the ring member 9 or container 3 via a friction fit or a snap-fit connection. In some embodiments, the cover 20 may connect to the container 3 even when the teat 18, sipping straw 24, or spoon 30 is not connected to the container, so as to function as a lid for the container 3. The cover 20 may have indentations 26 which enable it to be easily gripped for removal.

In the embodiment shown in FIG. 6 the cover 20 is configured to connect to the ring member 9. More particularly, the cover 20 is configured to removably snap-fit to the outside of the connection flange 13 of the ring member 9. The ring member 9 comprises a groove 23 on the outside of the connection flange 13 which is configured to receive a lip of the cover 20. In other embodiments the cover 20 may form a friction fit or snap-fit to the teat member 18 or container 3, rather than the ring member 9. In embodiments in which there is no ring member the cover 20 may fit to the teat member 18 or container 3, for example by way of a threaded connection, friction fit or snap-fit. In other embodiments, the cover may be hingedly connected to the ring member 9 or teat member 18 and either integrally formed therewith or assembled thereto.

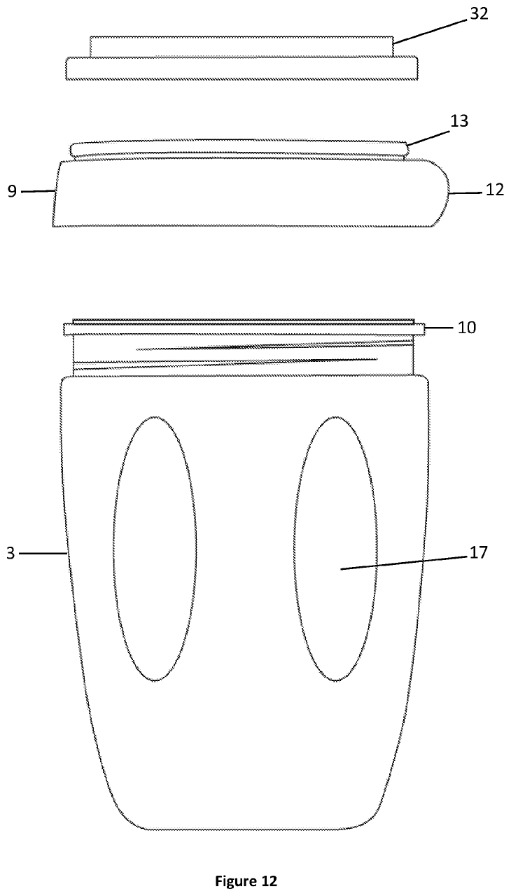

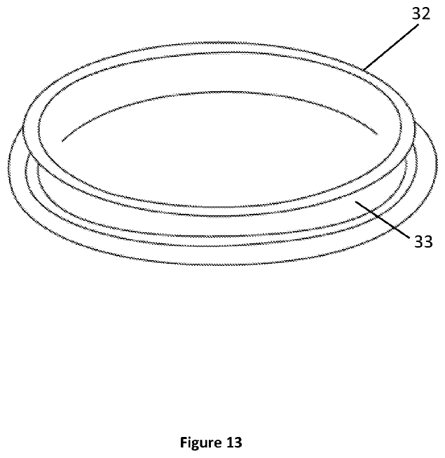

In some forms of the technology, the breast pump and feeding kit may also include a container lid 32 as shown in FIGS. 12 and 13. Once the breast milk has been obtained using the breast pump 1, the cup member 2 may be removed from the container 3, and the container lid 32 may be connected to the container 3. The container lid 32 enables the breast milk to be stored in the container 3 until it is ready for use. The container lid 32 may seal the opening of the container 3 to keep the milk fresh and to prevent anything entering the container 3 and contaminating the milk. It also ensures that the milk does not leak or spill out of the container 3. The container lid 32 may be made of a flexible material, such as silicone, rubber, or a flexible plastic. Alternatively, the container lid 32 may be formed of a rigid material, such as polypropylene, polyethylene, polycarbonate, a suitable metal, or glass. The lid 32 may also be formed from a combination of rigid and flexible materials. The container lid 32, as shown in FIG. 13, may comprise a groove 33 similar to that on the teat 18 or cup member 2, which engages with flange 13 on the ring member in a similar manner as described above. In alternate forms, the container lid 32 may directly attach to the container 3 (with the attachment means removed), for example by screwing to the threaded portion on the rim of the container 3.

A plurality of any of the components may be provided in the breast pump and feeding kit, in some embodiments. For example, the breast pump kit may comprise two (or more) ring members 9. One ring member 9 may then remain attached to a cup member 2 and a second ring member 9 may remain attached to the teat 18, sipping straw 24, or spoon 30. This allows the removal of the cup member 2 by disconnecting the ring member 9 from the container 3 and then connection of the teat 18, sipping straw 24, or spoon 30 to the container 3 by connecting the second ring member 9 to the container 3.

There are many ways in which the container 3 is configured to be removably and sealingly connected to a teat 18, sipping straw 24, or spoon 30 for feeding breast milk from the container to a baby, in the various embodiments of the present invention. Additionally, there are many different options for making the connection between the container and the cup member. Unless the context or description requires otherwise, any manner in which the cup member and container are able to be connected is also a manner in which the container may be configured to be removably and sealingly connected to the teat 18, sipping straw 24, or spoon 30, in various embodiments of the present invention.

In one form of the technology (not shown) the ring member 9 may engage with the inside of the top rim of the container 3 rather than the outside. For example, the container 3 may comprise an internal screw thread provided on an inner surface of the container 3. The ring member 9 may comprise an external screw thread provided on an outer surface of the ring member 9 corresponding to the internal screw thread of the container 3.

In another embodiment, the ring member 9 is able to separate into two half-circle portions which are configured to connect via a clip mechanism. The container 3 and the teat 18 may be placed adjacent to each other and the ring member clipped together around the container 3 and the teat 18, to connect the container 3 and the teat 18. In one example, one of the connections between the half-circle portions may be a hinged connection. The ring member 9, once closed on the container 3 and the teat 18, may hold the container 3 and teat 18 together tightly to create a secure and sealed connection.

Other embodiments of the present invention (all not shown) are described below.

Some embodiments of the invention are provided with further alternative connection means that enable the container 3 to connect to the teat 18 or cup member 2. In one form of the technology, the top rim of the container 3 may have a size and a shape which enables it to engage with a bottom rim of the teat 18. The container 3 may be deformed by the user so that it can enter the opening formed by the bottom rim of the teat 18 and then released back to its original shape to engage with the inside of the bottom rim of the teat 18.

In one form of the technology, the container 3 may connect to the teat 18 via a magnetic connection. The top rim of the container 3 may comprise one or more magnets which are configured to be attracted to one or more magnets located in the bottom rim of the teat 18.

In another form of the technology, the container 3 may comprise a spring-loaded clip mechanism. The mechanism may comprise a ridge forming part of the top rim of the container 3 that engages with a complementary groove on the bottom rim of the teat 18. The ridge may be hinged such that pushing a button will disengage the ridge from the groove and allow removal of the teat 18.

In an alternative form of the technology, the top rim of the container 3 may comprise one or more spring loaded buttons or pins, and the bottom rim of the teat 18 may comprise apertures with corresponding shapes. The buttons may be pushed radially inwardly so that the rim of the container 3 can enter the opening of the bottom rim of the teat 18 and when the buttons are aligned with the apertures the buttons will release and will engage the corresponding apertures to prevent the container 3 and the teat 18 disconnecting.

In some embodiments, the top rim of the container 3 may be formed of a substantially rigid biocompatible material such as polypropylene, polycarbonate, polyethylene, nylon, a metal suitable for contact with food such as aluminium or stainless steel, or glass. The rigid material may be overmoulded to the deformable elastic material that forms the rest of the container. The rigid material may comprise a screw thread that can connect with a corresponding screw thread on the teat 18 or the ring member 9. In alternative embodiments, the rigid material may comprise one or more protrusions, ridges and/or hooks or the like which are able to form a releasable snap-fit connection with corresponding protrusions or ridges on the teat 18 or ring member 9.

In alternative forms of the technology, the top rim of the container 3 is formed from a material that may effectively adhere to the bottom rim of the teat 18, e.g. by way of significant static friction. In some examples, the container 3 may be formed from a material (e.g. silicone) that has properties that allow it to effectively adhere to the material of the teat 18. For example, one or both of the container 3 and teat 18 may have a surface finish/texture that gives it a tendency to cling or stick to either of the container 3 or teat 18.

Reference to any prior art in this specification is not, and should not be taken as, an acknowledgement or any form of suggestion that that prior art forms part of the common general knowledge in the field of endeavour in any country in the world. All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

Aspects of the present invention have been described by way of example only and it should be appreciated that modifications and additions may be made thereto without departing from the scope thereof.

1-44. (canceled)

45. A breast pump and feeding kit for the pumping and feeding of breast milk, the kit comprising:
a cup member having a breast engaging portion configured to seal to a breast;
a container; and
a teat,
wherein the cup member and the container are configured to be removably and sealingly connected together and the container is configured to be deformed by a user and released to create suction on the breast to pump breast milk from the breast into the container; and
wherein the teat and the container are configured to be removably and sealingly connected together for feeding breast milk from the container to a baby.

46. The breast pump and feeding kit of claim 45, further comprising a ring member configured to connect the cup member to the container, the ring member also being configured to connect the teat to the container in place of the cup member.

47. The breast pump and feeding kit of claim 45, wherein the ring member is substantially rigid.

48. The breast pump and feeding kit of claim 46, wherein the container comprises a rim comprising a screw thread to connect to a complementary screw thread on the ring member.

49. The breast pump and feeding kit of claim 46, wherein the cup member and the ring member each comprises an end groove configured to fit to the container and seal therewith.

50. The breast pump and feeding kit of claim 46, wherein the ring member comprises a connection flange and wherein the cup member and the teat each comprises a circumferential groove configured to receive the connection flange.

51. The breast pump and feeding kit of claim 45, wherein the breast pump and feeding kit further comprises a teat cover configured to connect to and at least partially cover the teat.

52. The breast pump and feeding kit of claim 45 wherein one or more of the cup member, the teat and the container are formed from silicone.

53. The breast pump and feeding kit of claim 45, further comprising a spoon attachment configured to be removably and sealingly connected to the container, the spoon attachment comprising a spoon formation and an aperture extending from an interior surface of a connecting portion of the spoon attachment to the spoon formation to allow a liquid food to flow from the container to the spoon formation.

54. The breast pump and feeding kit of claim 45, further comprising a sipping straw attachment configured to be removably and sealing connected to the container, the sipping straw attachment comprising a straw and a nozzle.

55. The breast pump and feeding kit of claim 45, further comprising a container lid configured to connect to and close the container.

56. A breast pump comprising:
a cup member having a breast engaging portion configured to seal to a breast;
a container configured to be connected to the cup member; and
a ring member configured to removably and sealingly connect the cup member to the container,
wherein the container is configured to be deformed by a user and released to create suction on the breast to pump breast milk from the breast into the container.

57. The breast pump of claim 56, wherein the ring member is substantially rigid.

58. The breast pump of claim 56, wherein the container comprises a screw thread to connect to a complementary screw thread on the ring member.

59. The breast pump of claim 58, wherein the ring member comprises a tab extending from a side of the ring member, the tab configured to assist the user in screwing the ring member on to or off of the container.

60. The breast pump of claim 56, wherein one of the ring member and the cup member comprises a connection flange and the other of the ring member and the cup member comprises a circumferential groove configured to receive the connection flange.

61. The breast pump of claim 56, wherein the cup member seals to a rim of the container when the cup member is connected to the container.

62. The breast pump of claim 61, wherein the cup member comprises an end groove configured to fit to the rim of the container and seal thereto.

63. A breast pump of claim 56, wherein the cup member comprises a neck portion connected to the breast engaging portion, the neck portion being narrower than the breast engaging portion.

64. The breast pump of claim 56, wherein the cup member comprises a breast engaging portion configured to seal to a breast about an opening of the breast engaging portion, wherein the breast engaging portion opens at an oblique angle to an axis of the container.
